(19) United States
(12) Patent Application Publication
(10) Pub. No.: US 2025/0049663 A1
(43) Pub. Date: Feb. 13, 2025

(54) SUNSCREEN OR DAILY CARE COMPOSITION COMPRISING BIS-ETHYLHEXYLOXYPHENOL METHOXYPHENYL TRIAZINE

(71) Applicant: BASF SE, Ludwigshafen am Rhein (DE)

(72) Inventors: Myriam SOHN, Grenzach-Wyhlen (DE); Stanislaw KRUS, Grenzach-Wyhlen (DE)

(21) Appl. No.: 18/797,592

(22) Filed: Aug. 8, 2024

Related U.S. Application Data
(60) Provisional application No. 63/531,850, filed on Aug. 10, 2023.

(51) Int. Cl. A61K 8/35; A61Q 17/04

(52) U.S. Cl. CPC A61K 8/35; A61Q 17/04; A61K 2800/436; A61K 2800/592

(57) ABSTRACT

The present invention relates to a sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone), and
(iii) titanium dioxide;
wherein the sunscreen or daily care composition is free of the UV filters from the group consisting of 4,4′,4″-(1,3,5-triazin-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone).

### CROSS-REFERENCE TO RELATED APPLICATIONS

This application claims benefit of U.S. Application No. 63/531,850, filed Aug. 10, 2023, which is incorporated herein by reference in its entirety.

### TECHNICAL FIELD

The present application relates to a sunscreen or daily care composition comprising bis-ethylhexyloxyphenol methoxyphenyl triazine (Bemotrizinol), (ii) 1-(4-(1,1-dimethylethyl)phenyl)-3-(4-methoxyphenyl)propane-1,3-dione (avobenzone) and (iii) titanium dioxide.

### BACKGROUND

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter, UV-B filter and broadband filters. These filters are added to sunscreen or daily care compositions. The UV filters are either organic or inorganic, particulate or non-particulate compounds, of which all have a high absorption efficacy in the UV-light range. In general, UV light can be divided into UV-A radiation and UV-B radiation. Depending on the position of the absorption maxima, UV-filters are divided into UV-A and UV-B filters. In case a UV-filter absorbs both, UV-A and UV-B light, it is referred to as a broadband absorber. Since broadband UV filters provide protection against UVA and UVB radiation, it is preferred to add these kind of UV filters into sunscreen or daily care composition to ensure a broad and efficient UV protection foundation over the whole UV range.

Generally, sunscreen and daily care compositions comprise a multitude of UV filters and further components to provide optimal protection across the entire UV-A and UV-B range. However, this approach can come with several disadvantages. For one, the sourcing of a complex mixture of different UV filters can be daunting and expensive, thus also driving up the price of the final product. Additionally, specific UV filter combinations might result in photounstable formulations or components, for example the combination of avobenzone and octinoxate has been found to result in the photodecomposition of avobenzone and further in a hetero-cycloaddition of both molecules resulting in a loss of UV protection. Finally, some of the most commonly used UV filters are under suspicion for their health and environmental risks. For example, the commonly used UV filters octocrylene, homosalate, octinoxate, and oxybenzone are under suspicion to exhibit hormone-like behavior in the human body.

Therefore, it is desirable to develop sunscreen and daily care compositions that can provide good protection from UV radiation and have excellent overall sunscreen/daily care composition properties regarding skin feel, glossiness and stickiness while requiring as few different UV filters as possible.

It is thus an objective of the present invention to provide a composition that can be used for sunscreen products with sun protection levels spanning from low to high SPF (SPF from 6 to more than 50), with a good UVA protection of UVAI/UV ratio of at least 0.7, low glossiness, and low stickiness while requiring a small number of different UV filters.

There are only few UV filters that have the ability to cover the entire range of harmful UV light irradiation without any known negative impact on the environment or human health. Bis-ethylhexyloxyphenol methoxyphenyl triazine (Bemotrizinol, compound (1)) (abbreviation: BEMT) stands out in this group of broadband UV filters since it exhibits a wide and high sun protection band in the UV-A and UV-B range with a E1,1 (extinction 1% filter at an optical thickness of 1 cm) of >730 in the UVB and higher than 800 in the UVA range while having a strong photostability and a good health and environmental profile.

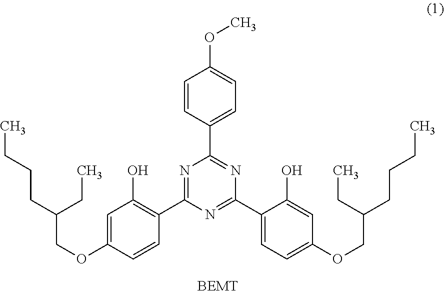 [SMILES: CCCCC(CC)COc1ccc(-c2nc(-c3ccc(OC)cc3)nc(-c3ccc(OCC(CC)CCCC)cc3O)n2)c(O)c1]

A useful UVB filter is titanium dioxide Together these two UV filters can provide excellent UV protection across the entire relevant UV light range. However, sunscreen compositions comprising BEMT and titanium dioxide have been found to be sticky which can result in an unpleasant feel on the skin.

### SUMMARY OF THE INVENTION

Surprisingly, it was now discovered that this issue is solved by adding 1-(4-(1,1-dimethylethyl)phenyl)-3-(4-methoxyphenyl)propane-1,3-dione (avobenzone) to sunscreen compositions comprising BEMT and titanium dioxide. The addition of avobenzone to a sunscreen comprising BEMT as broadband UV filter leads to a significant reduction of the stickiness/adhesiveness of the sunscreen composition. Further surprisingly, typical issues related to the use of avobenzone in sunscreens such as a lack of photostability of avobenzone and an increased glossiness of compositions with avobenzone do not materialize when avobenzone is combined with BEMT and titanium dioxide—as long as the compositions is free of all of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone).

Therefore, the present invention relates in a first aspect to a sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone), and
(iii) titanium dioxide;
wherein the sunscreen or daily care composition is free of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone). This sunscreen or daily care composition provides a high level of sun protection while also showing lower gloss and reduced stickiness compared to alternative formulations. According to one embodiment of the first aspect of the invention, the sunscreen or daily care composition has a SPF of at least 30 and an UVA1/UV ratio of at least 0.7.

In a second aspect, the present invention relates to a method for reducing the adhesiveness of a sunscreen or daily care composition comprising (i) bis-ethylhexyloxyphenol methoxyphenyl triazine (bemotrizinol) by adding (ii) 1-(4-(1,1-dimethylethyl)phenyl)-3-(4-methoxyphenyl)propane-1,3-dione (avobenzone) to the sunscreen or daily care composition.

In a third aspect, the present invention relates to a method for reducing the glossiness of a sunscreen or daily care composition comprising (ii) 1-(4-(1,1-dimethylethyl)phenyl)-3-(4-methoxyphenyl)propane-1,3-dione (avobenzone) by adding (i) bis-ethylhexyloxyphenol methoxyphenyl triazine (bemotrizinol) to the sunscreen or daily care composition.

The present invention will be described with respect to particular embodiments and with reference to certain examples, but the invention is not limited thereto, and it is only defined by the appending claims.

### BRIEF DESCRIPTION OF FIGURES

FIG. 1: Comparison of the stickiness of two comparative compositions with one inventive sunscreen composition;

FIG. 2: Comparison of the glossiness of two comparative compositions with one inventive sunscreen composition.

### DEFINITIONS

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20% preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.10% by weight, based on the total weight of the composition. Furthermore, it is preferred that the composition according to the present invention does not comprise said compounds or group of compounds in an amount of more than 0.05% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “cosmetic composition” refers to any topical product which is intended to be applied to the human body for cleansing, beautifying, promoting attractiveness, altering the appearance, or protecting the skin without affecting the body's structure or function. Possible cosmetic compositions within the meaning of the invention comprise leave-on and rinse-off products such as face and body care including daily care compositions with or without UV protection and sunscreen or sunscreen compositions, hair care products such as shampoos, conditioners, shower gels, make-up compositions and similar compositions.

The term “sunscreen composition” or “sunscreen” refers to any topical product which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product which is intended to be used daily without or with a UV protection. In case UV protection is included, it absorbs and may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. sunscreen preparations and skin care preparations.

Suitable skin care preparations are e.g., moisturizing, anti-ageing, refining, and lifting preparations. The cited leave-on compositions can be in the form of creams, ointments, pastes, foams, gels, lotions, powders, make-ups, sprays, sticks or aerosols. Daily-care compositions with UV protection protect against damages on human skin by UV light.

The term “UV filter” or “ultraviolet filter” as used herein refers to organic or inorganic compounds, which can absorb and may further reflect and scatter UV radiation caused by sunlight. UV-filter can be classified based on their UV absorbance profile as UV-A, UV-B, or broadband filters. Preferably, the term “UV filter” comprises or consists of any UV filter as defined in the Annex VI (version of 11.11.2022) of the Regulation (EC) No 1223/2009 of the European Parliament and of the Council.

Water soluble UV filters have a solubility in water of at least 2% by weight, preferably at least 3% by weight, more preferably at least 5% by weight.

Oil soluble filters have a solubility in specific emollients of at least 2% by weight, preferably at least 3% by weight, more preferably at least 5% by weight.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. Alternatively, or additionally, the emollients improve the distribution of the product on the skin. Emollients can be divided into different groups depending on their chemistry and/or polarity index.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a dosage of 2 milligrams per square centimeter (mg/cm2).

The definition of “broadband” protection (also referred to as broad-spectrum or broad protection) may be based for example on the “critical wavelength” or the UVA1/UV ratio or the UVA-PF. For broadband coverage, UV-B and UV-A protection must be provided. According to the US requirements, a critical wavelength of at least 370 nm is required for achieving broad spectrum protection. In the proposed order (OTC000008) published Sep. 24, 2021, the FDA proposed to add an additional requirement to pass the broad-spectrum test, the product shall meet a UVA1/UV ratio of at least 0.7. Furthermore, it is recommended by the European Commission that all sunscreens or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at minimum at 370 nm.

The term “administration” refers to the application of a sunscreen or daily care composition to the skin of a person.

### Particular Embodiments of the Invention

The first aspect of the present invention relates to a sunscreen or daily care composition comprising the UV filter combination of (i) bis-ethylhexyloxyphenol methoxyphenyl triazine (bemotrizinol), (ii) 1-(4-(1,1-dimethylethyl)phenyl)-3-(4-methoxyphenyl)propane-1,3-dione (avobenzone), and (iii) titanium dioxide wherein the sunscreen or daily care composition is free of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone). Such a sunscreen or daily care composition provides UV protection across the entire harmful UV light range while exhibiting a reduced stickiness and glossiness.

According to one embodiment of the invention, the sunscreen or daily care composition comprises Phenylbenzimidazole Sulfonic Acid (PBSA/Enzulizole) as further UV filter component. Enzulizole can be used for sunscreen or daily care compositions that require a high sun protection factor and can help to reduce whitening.

According to one preferred embodiment, Ensulizole is used as a salt. According to a further preferred embodiment of the invention, Ensulizole is used as a salt of an organic nitrogen compound, in particular as a salt of an amine base compound, in particular as tromethamine Ensulizole, triethanolamine Ensulizole, tetrahydroxypropyl ethylenediamine, or arginine Ensulizole. These salts protect Ensulizole from crystallization. The statement that Ensulizole is used as a salt, in particular as a salt of an amine base compound, in particular as tromethamine Ensulizole, triethanolamine Ensulizole, tetrahydroxypropyl ethylenediamine, or arginine Ensulizole means that the Ensulizole is present in form of a dissolved salt in the sunscreen or daily care composition. This can be achieved either by providing the salt form of Ensulizole prior to adding it to the sunscreen or daily care composition or in-situ in the sunscreen or daily care composition by providing the neutralizing compound, in particular tromethamine, triethanolamine, tetrahydroxypropyl ethylenediamine, or arginine to the sunscreen or daily care composition separately.

According to a preferred embodiment of the invention, the sunscreen or daily care composition comprises from 0.5 to 8.0 wt. % Enzulizole, in particular from 0.5 to 4.0 wt. % or from 1.0 to 3.5 wt. % or from 1.5 to 3.0 wt. % or from 2.0 to 3.0 wt. %, in particular 0.5 wt. %, 1.0 wt. %, 1.5 wt. %, 2.0 wt. %, 2.5 wt. %, 3.0 wt. %, 3.5 wt. % or 4.0 wt. % Enzulizole, each based on the total weight of the sunscreen or daily care composition. The weight ratio is based on Enzulizole without the respective counterion in case that Enzulizole is used as a salt.

According to a preferred embodiment of the present invention, the sunscreen or daily care composition is free of any further organic UV filter. The combination of UV filters (i)-(iii) in the present invention is sufficient to provide UV protection across the entire UV range. In case Enzulizole is present in the inventive sunscreen or daily care composition, it is preferred that the composition is free of any further organic UV filter. Limiting the number of different UV filters present in the composition also reduces the risk for unintended interactions or reactions of these different components. It also reduces the risk for any allergic reactions due to other types of UV filters. The term “organic UV filter” is defined as any organic compound that is a UV filter as listed in the Annex VI (version of 11.11.2022) of the Regulation (EC) No 1223/2009 of the European Parliament and of the Council.

According to one preferred embodiment of the invention, the sunscreen or daily care composition is free of zinc oxide. In another preferred embodiment, the sunscreen or daily care composition comprises zinc oxide. Zinc oxide can be used to further increase the SPF of the inventive composition.

If zinc oxide is present in the sunscreen or daily care composition, it is preferably that the zinc oxide is coated. Preferably, the zinc oxide is coated with inorganic and/or organic surface coatings.

In a preferred embodiment of the present invention, the zinc oxide is coated with inorganic surface coating selected from the group consisting of sodium hexametaphosphate (NaPO3), sodium meta-phosphate (NaPO3)n, silicon dioxide (SiO2) (also: Silica, e.g. CAS-No.: 7631-86-9), iron oxide (Fe2O3), and mixtures thereof. It is to be understood that these inorganic surface coatings can be present on their own, in combination and/or in combination with organic surface coatings.

In a preferred embodiment of the present invention, the zinc oxide is coated with organic surface coatings selected from the group consisting of fatty acids such as stearic acid or lauric acid, dimethylpolysiloxane (also: dimethicone), methylpolysiloxane (methicone), simethicone (a mixture of dimethylpolysiloxane with an average chain length of about 200 to about 350 dimethylsiloxane units and silica gel), triethoxycaprylylsilane, octyltrimethoxysilane, and mixtures thereof. It is to be understood that these organic surface coatings can be present on their own, in combination and/or in combination with inorganic surface coatings. Preferably, the zinc oxide according to the present invention is uncoated or coated with dimethicone, methicone, or triethoxycaprylylsilane, most preferably the zinc oxide according to the present invention is surface coated with triethoxycaprylylsilane.

According to one embodiment of the invention, the sunscreen or daily care composition comprises from 0.5 to 25.0 wt. % zinc oxide, based on the total weight of the sunscreen or daily care composition. Other advantageous amounts of zinc dioxide in the inventive sunscreen or daily care composition are 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10.0 wt. %, 10.5 wt. %, 11.0 wt. %, 11.5 wt. %, 12.0 wt. %, 12.5 wt. %, 13.0 wt. %, 13.5 wt. %, 14.0 wt. %, 14.5 wt. %, 15.0 wt. %, 15.5 wt. %, 16.0 wt. %, 16.5 wt. %, 17.0 wt. %, 17.5 wt. %, 18.0 wt. %, 18.5 wt. %, 19.0 wt. %, 19.5 wt. %, 20.0 wt. %, 20.5 wt. %, 21.0 wt. %, 21.5 wt. %, 22.0 wt. %, 22.5 wt. %, 23.0 wt. %, 23.5 wt. %, 24.0 wt. %, 24.5 wt. %, 25.0 wt. % as well as all values in between, for example 2.0 wt. % to 25.0 wt. %, 2.5 wt. % to 24.5 wt. %, 3.0 to 24.0 wt. %, 3.5 wt. % to 23.5 wt. %, 4.0 wt. % to 23.0 wt. %, 4.5 wt. % to 22.5 wt. %, 5.0 wt. % to 22.0 wt. %, 5.5 wt. % to 21.5 wt. %, 6.0 wt. % to 21.0 wt. %, 6.5 wt. % to 20.5 wt. %, 7.0 wt. % to 20.0 wt. %, 7.5 wt. % to 19.5 wt. %, 8.0 wt. % to 19.0 wt. %, 8.5 wt. % to 18.5 wt. %, 9.0 wt. % to 18.0 wt. %, 9.5 wt. % to 17.5 wt. %, 10.0 wt. % to 17.0 wt. %, 10.5 wt. % to 16.5 wt. %, 11.0 wt. % to 16.0 wt. %, 11.5 wt. % to 15.5 wt. %, 12.0 wt. % to 15.0 wt. %, 12.5 wt. % to 14.5 wt. %, 13.0 wt. % to 14.0 wt. %, each based on the total weight of the sunscreen or daily care composition.

The sunscreen or daily care composition of the present invention comprises (iii) titanium dioxide. In a preferred embodiment of the present invention, the titanium dioxide (iii) is a nano material.

In this connection it is to be understood that the term “nano material” follows the recommendation of the European Commission 2011/696/EU. Accordingly, in a nano material 50% of particles, based on a number-based size distribution, are smaller than 100 nm, including constituent particles in aggregates or agglomerates. In another preferred embodiment of the present invention, the titanium dioxide is a non-nano material. Accordingly, in a non-nano material more than 50% of the particles, based on a number-based size distribution, are equal or larger than 100 nm.

In a preferred embodiment of the present invention, the titanium dioxide has a number-average elementary particle diameter of less than 1000 nm, preferably of less than 200 nm, more preferably of less than 100 nm, and in particular less than 50 nm. It is further preferred that the titanium dioxide has a number-average elementary particle diameter of 0.1 to less than 1000 nm, more preferably of 1 to 100 nm, and in particular of 2 to 50 nm. The number-average elementary particle diameter of said titanium dioxide particles may be determined by any known method in the art such as transmission electron microscopy (TEM).

According to a preferred embodiment of the invention, the titanium dioxide is coated. Uncoated titanium dioxide is photocatalytic and should be avoided in sunscreens. Preferably, the titanium dioxide is a coated titanium dioxide which is a pigment that has undergone one or more surface treatments of chemical, electronic, mechanochemical and/or mechanical nature preferably with compounds selected from the group consisting of amino acids, beeswax, fatty acids, fatty alcohols, anionic surfactants, lecithins, sodium, potassium, zinc, iron salts of fatty acids (stearic acid, metal alkoxides (of titanium), polyethylene, silicones (such as dimethicone, cyclomethicone, polysilicone, simethicone, dimethicone/methicone copolymer and/or cyclopentasiloxane), proteins (collagen, elastin), alkanolamines, silicon oxides, metal oxides, sodium hexametaphosphate, silica and mixtures thereof, more preferably wherein the titanium oxide is coated with stearic acid, silica, dimethicone and mixtures thereof.

Preferably, the coated titanium dioxide pigments are titanium oxides that have been coated:
with silica, such as the product Sunveil from the company Ikeda, the product Eusolex T-AVO from the company Merck, and the product Sunsil TIN 50 from the company Sunjin;
with silica and iron oxide, such as the product Sunveil F from the company Ikeda; and Microtitanium Dioxide MT 100 SA from the company Tayca and Tioveil from the company Croda;
with iron oxide and iron stearate, such as the product Microtitanium Dioxide MT 100 F from the company Tayca;
with silica and a silicone, such as the product UV-Titan X 195 from the company Sachtleben Pigments, and Parsol TX from the company DSM Nutritional Products;
with silica and cetyl phosphate such as the product Eusolex T-easy from Merck;
with UV Titan M 262 (with dimethicone) from the company Sachtleben Pigments; Eusolex T-2000 (with simethicone) from Merck;
with triethanolamine, such as the product STT-65-S from the company Titan Kogyo;
with stearic acid, such as the product Tipaque TTO-55 (C) from the company Ishihara;
with sodium hexametaphosphate, such as the product Microtitanium Dioxide MT 150 W from the company Tayca;
with silicone such as trimethoxycaprylylsilane or simethicone;
with octyltrimethylsilane, notably sold under the trade name T 805 by the company Degussa Silices;
with a polydimethylsiloxane, notably sold under the trade name 70250 Cardre UF TiO2SI3 by the company Cardre;
anatase/rutile TiO2 treated with a polydimethylhydrogenosiloxane, notably sold under the trade name Microtitanium Dioxide USP Grade Hydrophobie by the company Color Techniques. Mention may also be made of titanium dioxide pigments doped with at least one transition metal such as iron, zinc or manganese and more particularly manganese. Preferably, said doped pigments are in the form of an oily dispersion. The oil present in the oily dispersion is preferably chosen from triglycerides including those of capric/caprylic acids.

The sunscreen or daily care composition of the present invention comprises (i) BEMT and (ii) avobenzone. In general, the weight ratio of these two components can vary broadly. In a preferred embodiment of the invention, the weight ratio of (i) bis-ethylhexyloxyphenol methoxyphenyl triazine (bemotrizinol) to (ii) 1-(4-(1,1-dimethylethyl)phenyl)-3-(4-methoxyphenyl)propane-1,3-dione (avobenzone) is from 1:3 to 3:1. Therefore, the total weight amount of BEMT in the sunscreen or daily care composition in this preferred embodiment is at most three times the total weight amount of avobenzone in the sunscreen or daily care composition and the total weight amount of avobenzone in the composition is at most three times the total weight amount of BEMT. It is also possible that the weight ratio of (i) bis-ethylhexyloxyphenol methoxyphenyl triazine (bemotrizinol) to (ii) 1-(4-(1,1-dimethylethyl)phenyl)-3-(4-methoxyphenyl)propane-1,3-dione (avobenzone) is selected from 1:2.5 to 2.5:1, from 1:2 to 2:1, from 1:1.5 to 1.5:1 or 1:1.

According to another preferred embodiment of the invention, the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, butyloctyl salicylate and combinations thereof. In one preferred embodiment, the sunscreen or daily care composition comprises butyloctyl salicylate. In another preferred embodiment, the sunscreen or daily care composition comprises butyloctyl salicate but is free of all of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, and polyester-25. However, in another preferred embodiment of the invention, the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, and mixtures thereof and is free of butyloctyl salicylate.

According to a preferred embodiment of the invention, the sunscreen or daily care composition comprises
(i) bis-ethylhexyloxyphenol methoxyphenyl triazine (bemotrizinol) in an amount of 0.5 to 6 wt. %;
(ii) 1-(4-(1,1-dimethylethyl)phenyl)-3-(4-methoxyphenyl)propane-1,3-dione (avobenzone) in an amount of 1 to 5 wt. %;
(iii) titanium dioxide in an amount of 2 to 25 wt. %;
each based on the total weight of the sunscreen or daily care composition.

According to a more preferred embodiment, the sunscreen or daily care composition comprises
(i) bis-ethylhexyloxyphenol methoxyphenyl triazine (bemotrizinol) in an amount of 2.5 to 6 wt. %;
(ii) 1-(4-(1,1-dimethylethyl)phenyl)-3-(4-methoxyphenyl)propane-1,3-dione (avobenzone) in an amount of 2.5 to 5 wt. %;
(iii) titanium dioxide in an amount of 2 to 25 wt. %;
each based on the total weight of the sunscreen or daily care composition.

Other advantageous amounts of bis-ethylhexyloxyphenol methoxyphenyl triazine (bemotrizinol) in the inventive sunscreen or daily care composition are 2.5 wt. %, 3.0 wt. %, 3.5 wt. %, 4.0 wt. %, 4.5 wt. %, 5.0 wt. %, 5.5 wt. %, 6.0 wt. %, each based on the total weight of the sunscreen or daily care composition as well as any values in between, for example 2.5 wt. % to 6.0 wt. %, 3.0 wt. % to 5.5 wt. %, 3.5 wt. % to 5.0 wt. %, 4.0 to 4.5 wt. %, each based on the total weight of the sunscreen or daily care composition. Other advantageous amounts of 1-(4-(1,1-dimethylethyl)phenyl)-3-(4-methoxyphenyl)propane-1,3-dione (avobenzone) in the inventive sunscreen or daily care composition are 2.5 wt. %, 3.0 wt. %, 3.5 wt. %, 4.0 wt. %, 4.5 wt. %, 5.0 wt. %, each based on the total weight of the sunscreen or daily care composition as well as any values in between, for example 2.5 wt. % to 5.0 wt. %, 3.0 wt. % to 4.5 wt. %, 3.5 wt. % to 4.0 wt. %, each based on the total weight of the sunscreen or daily care composition. Other advantageous amounts of titanium dioxide in the inventive sunscreen or daily care composition are 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10.0 wt. %, 10.5 wt. %, 11.0 wt. %, 11.5 wt. %, 12.0 wt. %, 12.5 wt. %, 13.0 wt. %, 13.5 wt. %, 14.0 wt. %, 14.5 wt. %, 15.0 wt. %, 15.5 wt. %, 16.0 wt. %, 16.5 wt. %, 17.0 wt. %, 17.5 wt. %, 18.0 wt. %, 18.5 wt. %, 19.0 wt. %, 19.5 wt. %, 20.0 wt. %, 20.5 wt. %, 21.0 wt. %, 21.5 wt. %, 22.0 wt. %, 22.5 wt. %, 23.0 wt. %, 23.5 wt. %, 24.0 wt. %, 24.5 wt. %, 25.0 wt. % as well as all values in between, for example 2.0 wt. % to 25.0 wt. %, 2.5 wt. % to 24.5 wt. %, 3.0 to 24.0 wt. %, 3.5 wt. % to 23.5 wt. %, 4.0 wt. % to 23.0 wt. %, 4.5 wt. % to 22.5 wt. %, 5.0 wt. % to 22.0 wt. %, 5.5 wt. % to 21.5 wt. %, 6.0 wt. % to 21.0 wt. %, 6.5 wt. % to 20.5 wt. %, 7.0 wt. % to 20.0 wt. %, 7.5 wt. % to 19.5 wt. %, 8.0 wt. % to 19.0 wt. %, 8.5 wt. % to 18.5 wt. %, 9.0 wt. % to 18.0 wt. %, 9.5 wt. % to 17.5 wt. %, 10.0 wt. % to 17.0 wt. %, 10.5 wt. % to 16.5 wt. %, 11.0 wt. % to 16.0 wt. %, 11.5 wt. % to 15.5 wt. %, 12.0 wt. % to 15.0 wt. %, 12.5 wt. % to 14.5 wt. %, 13.0 wt. % to 14.0 wt. %, each based on the total weight of the sunscreen or daily care composition.

According to a preferred embodiment of the invention, the sunscreen or daily care composition exhibits an adhesive force of less than 60 mN in an in-vitro adhesion test 100 s after application. The sunscreen or daily care composition preferably exhibits an adhesive force of less than 55 mN, less than 54 mN, less than 53 mN, less than 52 mN, less than 51 mN, less than 50 mN, less than 49 mN, less than 48 mN, less than 47 mN, less than 46 mN, less than 45 mN, less than 44 mN, less than 43 mN, less than 42 mN, less than 41 mN, less than 40 mN, less than 39 mN, less than 38 mN in an in-vitro adhesion test 100 s after application. The adhesive force is measured by using a texture analyzer (TA.XT plusC) which measures the force (in mN) required to lift a silicone specimen (resembling a human finger) off the surface of a sunscreen-covered skin substitute. The details of how to determine the adhesive force required in the adhesion test are provided in the experimental section.

According to one preferred embodiment of the invention, the sunscreen or daily care composition exhibits a relative adhesive force that is less than 80% of an untreated collagen membrane as control sample in an in-vitro adhesion test 100 s after application. Also preferred, the sunscreen or daily care composition exhibits a relative adhesive force that is less than 79%, less than 78%, less than 77%, less than 76%, less than 75%, less than 74%, less than 73%, less than 72%, less than 71%, less than 70%, less than 69%, less than 68%, less than 67%, less than 66%, less than 65%, less than 64%, less than 63%, less than 62%, less than 61%, less than 60%, less than 59%, less than 58%, less than 57%, less than 56%, less than 55%, less than 54%, less than 53%, less than 52%, less than 51%, less than 50%, less than 49%, less than 48%, less than 47%, less than 46%, less than 45% of an untreated collagen membrane as control sample in an in-vitro adhesion test 100 s after application. The adhesive force is measured by using a texture analyzer (TA.XT plusC) which measures the force (in mN) required to lift a silicone specimen (resembling a human finger) off the surface of a sunscreen-covered skin substitute. The details of how to determine the adhesive force required in the adhesion test are provided in the experimental section. The results obtained are compared to an untreated collagen membrane which is set to 100. The %-values indicate the adhesion value in mN obtained for a collagen membrane treated with the inventive sunscreen or daily care composition with the adhesion value in mN obtained for an untreated collagen membrane.

According to another embodiment, the sunscreen or daily care composition exhibits a reduced adhesiveness compared to a sunscreen or daily care composition comprising (i) bis-ethylhexyloxyphenol methoxyphenyl triazine (bemotrizinol) and (iii) titanium dioxide but being free of (ii) 1-(4-(1,1-dimethylethyl)phenyl)-3-(4-methoxyphenyl)propane-1,3-dione (avobenzone).

In another embodiment of the invention, the sunscreen or daily care composition has a SPF of more than 15 or more than 20 or more than 25. The SPF value can be determined by using a SPF calculation system for in-silico determination of the SPF, for example using the BASF sunscreen simulator (https://sunscreensimulator.basf.com/). The calculation method is further described in B. Herzog et al. Simulation of sunscreen performance, Pure Appl. Chem. 2015; 87(9-10):937-951. Alternatively, the SPF can be determined in-vivo (according to ISO 24444) or in-vitro. SPF in-vitro determination is based on the assessment of UV-transmittance through a thin film of sunscreen sample spread on roughened substrate which is transparent to UV. Suitable substrates are for example PMMA plates (an example of PMMA plates is the SB6 PMMA-plates (roughness Ra is around 4-5 μm, area 4.8×4.8 cm) from Helioscreen, Creil, France). The sunscreen formulation to be tested is applied on the PMMA plate with an amount of 1.2 mg/cm² for a plate with a roughness of Ra of 4-5 μm. This amount may differ if a plate with different roughness is used as substrate. Three plates and 5 measurements per plate are done per formulation. The formulation is distributed manually as equally as possible on the plate. The plates applied with formulation are then stored 30 minutes at room temperature (22° C.) and in a dark place prior the measurement.

In vitro transmission measurement may be performed from 290-400 nm with 1 nm steps with the Labsphere UV Transmittance Analyzer UV 2000S. The UV transmission spectrum is then acquired and the SPF in vitro values are calculated according to equation below (mean SPF of all 15 values (from the three plates and 5 measurements per plate))

$$SPF = \frac{{\sum }_{290 nm}^{400 nm} {s}_{er} ( λ ) \cdot S ( λ )}{{\sum }_{290 nm}^{400 nm} {s}_{er} ( λ ) \cdot S ( λ ) \cdot T ( λ )}$$

where, sₑᵣ(λ) is the erythema action spectrum and S(λ) is the spectral irradiance received from the UV source and T(λ) is the in vitro measured light transmittance.

It is further preferred that the sunscreen or daily care composition has a protection ratio UVA1/UV equal or superior to 0.7 after an irradiation of 4 MED (according to the FDA proposed order (OT0000008) issued Sep. 24, 2021).

It is further preferred that the sunscreen or daily care composition has a ratio UVA-PF/SPF superior or equal to 1/3 (according to the EC recommendation of Sep. 22, 2006 on the efficacy of sunscreen products and the claims made relating thereto), more preferred of more than 0.4. The UVA-PF can be measured in vivo (ISO24442), in vitro (ISO24443) or also be calculated in-silico, preferably using the BASF sunscreen simulator (https://sunscreensimulator.basf.com/).

In a preferred embodiment of the present invention, the sunscreen or daily care composition provides a photostability of more than 80%, preferably of more than 85%, and in particular of more than 90%. Preferably, the sunscreen or daily care composition provides a photostability after 10 MED, 20 MED, and/or 50 MED, of more than 80%, preferably of more than 85%, and in particular of more than 90%. The photostability of the compositions is preferably determined by applying the respective composition on roughened quartz plates (2 μl/cm²) and then irradiating the plates using an Atlas CPS device with a certain minimal erythemal dose (MED) irradiation, preferably 50 MED irradiation. Then, the plates are rinsed off with tetrahydrofuran and the rinsing solution is analyzed via HPLC to determine the recovery of the UV filters. An alternative preferred method for determining the photostability of the composition comprises the irradiation of sunscreen-covered PMMA plates and evaluation of the difference between the absorbance spectrum before and after irradiation.

In a particularly preferred embodiment, the sunscreen or daily care composition exhibits a reduced glossiness compared to a sunscreen or daily care composition comprising only one of (i) bis-ethylhexyloxyphenol methoxyphenyl triazine (bemotrizinol) and (ii) 1-(4-(1,1-dimethylethyl)phenyl)-3-(4-methoxyphenyl)propane-1,3-dione (avobenzone). The combination of BEMT and avobenzone results in a reduced glossiness compared to a similar composition comprising only BEMT and in particular compared to a similar composition comprising only avobenzone. The glossiness of the compositions can be determined with the method described in the experimental section.

The inventive sunscreen and daily care compositions preferably comprise additives, in particular selected from the group consisting of emulsifiers, emollients, further viscosity regulators (thickeners), sensory enhancers, adjuvants, preservatives, perfumes and combinations thereof.

Preferred O/W emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives such as polysorbate derivatives;
   inulin derivatives such as inulin lauryl carbamate;
glycerides of fatty acids such as glyceryl stearate, glyceryl stearate SE, glyceryl stearate citrate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate, ceteh-10 phosphate, C20-22 alkyl phosphate;
fatty acid esters of polyglyceryl such as polyglyceryl-3-diisostearate, polyglyceryl-2-dipolyhydroxystearate; polyglyceryl-10 stearate
oxyalkenylated fatty alcohol such as ceteareth-20, steareth-21, beneheth-25
oxyalkenylated fatty acid such as PEG-100 stearate
oxyalkenylated organomodified silicone/polysiloxane/polyalkyl/polyether copolymers and derivatives.
phospholipids based such as lecithin derivatives

Preferred emulsifiers to form a W/O emulsion include
glycerides of fatty acids such glyceryl oleate, sorbitan laurate
sorbitan esters such as sorbitan oleate
fatty acid esters of polyglyceryl such as polyglyceryl-3-diisostearate, polyglyceryl-2-dipolyhydroxystearate, polyglyceryl-4 isostearate
Oxyakenylated fatty alcohol such steareth-2
Oxyakenylated fatty acid such as PEG-30 dipolyhydroxystearate
organomodified silicone/polysiloxane/polyalkyl/polyether copolymers and derivatives such as cetyl dimethicone copolyol, cetyl PEG/PPG-10/1 Dimethicone, PEG-10 dimethicone

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C12-C15-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C6-C18 fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane, cyclic silicones.
hydrocarbons such as isododecane, isohexadecane

Preferred lipophilic thickeners include
fatty alcohols such as cetyl alcohol, cetearyl alcohol, stearyl alcohol; behenyl alcohol
fatty acids such as stearic acid; palmitic acid
fatty acid esters such as myristyl stearate, pentaerythrityl distearate, cetyl palmitate; Tribehenin, dextrin palmitate
waxes such as beeswax, carnauba wax, microcrystalline wax, ceresin, ozocerite; Oryza sativa bran Wax, sunflower wax
hydrogenated vegetable oil, hydrogenated castor oil, hydrogenated vegetable glycerides,
hydrogenated castor oil/sebacic acid copolymer
poly C10-30 alkyl acrylate
polyamide derivatives such as polyaminde-8
silica based derivatives such as silica, silica dimethyl silylate

### Hydrophilic Stabilizers/Thickeners

natural gums such as xanthan gum, tara gum, carrageenan
silicate derivatives such as magnesium aluminium silicates;
cellulose derivatives such as hydroxypropyl cellulose, microcrystalline cellulose
polyacrylic acid-based derivatives such as sodium polyacrylate, Acrylates/C10-30 Alkyl Acrylate Crosspolymer, carbomer, Acrylates/Beheneth-25 Methacrylate Copolymer, Hydroxyethyl Acrylate/Sodium Acryloyldimethyl Taurate Copolymer
starch derivatives such as hydroxypropyl starch phosphate

### SPF Boosters

### UV Protection Scattering Boosters

Styrene/Acrylates Copolymer
Microcrystalline cellulose
Calcium carbonate
Hydroxyapatite
silica

### Film Formers/Film Forming Enhancers

Polyvinylpyrrolidone based compounds such as Triacontanyl PVP, VP/hexadecane copolymer, VP/Eicosene Copolymer, hydrolyzed wheat protein/PVP crosspolymer
Hydrogenated Dimer Dilinoleyl/Dimethylcarbonate Copolymer
Diisostearoyl Polyglyceryl-3 Dimer Dilinoleate
Polyglyceryl-3 Stearate/Sebacate Crosspolymer
Polyacrylate-15
Oryza sativa bran Wax
Carnauba wax
Cellulose derivatives such as Methylcellulose, ethylcellulose
Hydrogenated Castor oil
Capryloyl Glycerin/Sebacic Acid Copolymer
Acrylates/Octylacrylamide Copolymer

Preferred sensory enhancers include
silica;
mica;
polymethylsilsesquioxane;
starch derivatives such as aluminum starch octenylsuccinate; rice starch
dimethicone derivatives;
boron nitride;
HDI/trimethylol hexyllactone crosspolymer.
perlite

Preferred humectants include
glycerin
Butylene glycol
Propylene glycol
beta-glucan
1,2-pentadiol
1,2-hexandiol
1,2-octandiol
1,2-decandiol
2-Methyl-1,3-propandiol.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
hydroxyacetophenone;
caprylhydroxymic acid;
propellants such as propane, butane, isobutene, dimethyl ether;
insect repellants such as butylacetylaminopropionate.

Preferred preservatives include the compounds listed in annex V of the Regulation 1223/2009/EC on Cosmetic Products, in particular
benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

It is preferably that the inventive sunscreen or daily care composition comprises two or more of the additives described above. In connection with the above preferred embodiments, it is to be understood that if the sunscreen or daily care composition comprises two or more additives, combinations of any of the additives as defined above are also part of the invention.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions, sticks, or in the form of a sprayable product.

The second aspect of the invention relates to a method for reducing the adhesiveness of a sunscreen or daily care composition comprising (i) bis-ethylhexyloxyphenol methoxyphenyl triazine (bemotrizinol) comprising the step of adding (ii) 1-(4-(1,1-dimethylethyl)phenyl)-3-(4-methoxyphenyl)propane-1,3-dione (avobenzone) to the sunscreen or daily care composition.

Preferably, the sunscreen or daily care composition comprising (i) bis-ethylhexyloxyphenol methoxyphenyl triazine (bemotrizinol) further comprises (iii) titanium dioxide and is free of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone).

It was discovered that the addition of avobenzone to a composition comprising BEMT results in a reduction of the stickiness of the composition. Preferably, the addition of avobenzone is combined with a respective reduction of BEMT.

All the statements made regarding the components and properties of the sunscreen or daily care composition with regard to the first aspect of the invention also apply mutatis mutandis to the second aspect of the invention.

The third aspect of the invention relates to a method for reducing the glossiness of a sunscreen or daily care composition comprising (ii) 1-(4-(1,1-dimethylethyl)phenyl)-3-(4-methoxyphenyl)propane-1,3-dione (avobenzone) comprising the step of adding (i) bis-ethylhexyloxyphenol methoxyphenyl triazine (bemotrizinol). Preferably, the sunscreen or daily care composition comprising (ii) 1-(4-(1,1-dimethylethyl)phenyl)-3-(4-methoxyphenyl)propane-1,3-dione (avobenzone) further comprises (iii) titanium dioxide and is free of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone).

It was discovered that the addition of BEMT to a composition comprising avobenzone results in a reduction of the glossiness of the composition. Preferably, the addition of BEMT is combined with a respective reduction of avobenzone.

All the statements made regarding the components and properties of the sunscreen or daily care composition with regard to the first aspect of the invention also apply mutatis mutandis to the third aspect of the invention.

The invention also relates to a sunscreen or daily care composition comprising a UV filter combination of (i) bis-ethylhexyloxyphenol methoxyphenyl triazine (INCI bis-ethylhexyloxyphenol methoxyphenyl triazine), (ii) 1-(4-(1,1-dimethylethyl)phenyl)-3-(4-methoxyphenyl)propane-1,3-dione (avobenzone) and (iii) titanium dioxide and being free of any further UV filter.

The invention also relates to a sunscreen or daily care composition comprising a UV filter combination of (i) bis-ethylhexyloxyphenol methoxyphenyl triazine (INCI bis-ethylhexyloxyphenol methoxyphenyl triazine), (ii) 1-(4-(1,1-dimethylethyl)phenyl)-3-(4-methoxyphenyl)propane-1,3-dione (avobenzone), (iii) titanium dioxide, and (iv) zinc oxide, and being free of any further UV filter.

The invention is further illustrated by the following examples.

### Examples

### Methods

### Stickiness (In Vitro Adhesion Test)

A collagen biomatrix film membrane served as skin substitute, its wall thickness is about 146 μm and it has a roughness Ra comparable to human skin. 2 mg/cm² of tested sunscreen was applied on the collagen biomatrix film membrane using a brush with synthetic bristles on a surface area on 5×6 cm. The collagen membrane substrate was fixed on a sample table maintained at 36° C. using double sided adhesive tape.

The sample table was located under a texture analyzer (TA.XT plusC) which measured the force (in mN) required to lift the silicone specimen (resembling a human finger) off the surface of the sunscreen-covered skin substitute.

The force was measured 20 times, one time every 10 seconds. Three measurements per formulation were done. The measured force is associated to the stickiness; the stickier a formulation, the higher is the measured force needed to remove the silicone specimen from the surface of the applied sunscreen; inversely, the lower the measured force, the less sticky is the formulation. The first value at t=0 s corresponds to the measurement of control of the untreated collagen membrane which is set to 100.

### Gloss

A collagen biomatrix (CBM) film membrane served as skin substitute, its wall thickness is about 146 μm and it has a roughness Ra comparable to human skin. The CBM is cut into a 5×5 cm sheet and adhered to the center of a plastic card using double sided adhesive tape. The adhered CBM is then wiped-off with ethanol-soaked cosmetic paper tissues. 2 mg/cm² of tested formulation was applied on the collagen biomatrix film membrane using a brush with synthetic bristles on the surface area of 5×5 cm and the card is positioned below the digital camera (“Canon S12” (ISO-80, f-number: F/1.8, shutter speed: 1/60 sec) with a curvature of 0.75-0.85). A round LED panel with a linear polarization filter and emitting warm white light is mounted approximately 40 cm above the measurement area. The digital camera is fixed on a tripod approximately 15 cm above the measurement area. A 90°-switchable linear polarization filter is mounted in front of the lens. This setup provides the means to take cross and parallel polarized images. The cross polarized image contains the information of the color of the probe and the parallel polarized image contains the information of the color plus gloss of the same probe. The image data can be specifically subtracted from one another to obtain the image containing only gloss of the tested probe. The resulting subtracted image is then evaluated using the image processing software Image J, which provides the grey values distribution which relates to the gloss intensity of the probe (the higher the mean grey value, the higher the gloss intensity). The gloss intensity of the collagen biomatrix (CBM) without the applied tested formulation is set to a value of 0 as reference point. The gloss intensity of tested formulation is given as a change to this reference point (CBM without formulation).

### Formulations

TABLE 1
Oil/Water formulations

| | | Comp. | Comp. | Inv. |
|---|---|---|---|---|
| | | formu- | formu- | formu- |
| | | lation 1 | lation 2 | lation 3 |
| Part | INCI | [wt. %] | [wt. %] | [wt. %] |
| A | Polyglyceryl-2- | 3.00 | 3.00 | 3.00 |
| | dipolyhydroxystearate, | | | |
| | lauryl glucoside, glycerin | | | |
| | Beheneth-25 | 1.00 | 1.00 | 1.00 |
| | Polyhydroxystearic acid | 1.00 | 1.00 | 1.00 |
| | C12-15 alkyl benzoate | 10.00 | 10.00 | 10.00 |
| | Dibutyl adipate | 10.00 | 10.00 | 10.00 |
| | Dicaprylyl carbonate | 5.00 | 5.00 | 5.00 |
| | Phenoxyethanol, Ethylhexyl | 1.00 | 1.00 | 1.00 |
| | glycerin | | | |
| | Avobenzone | — | 5.00 | 2.50 |
| | Bemotrizinol | 5.00 | — | 2.50 |
| | Titanium dioxide (nano, | 7.14 (6.00 | 7.14 (6.00 | 7.14 (6.00 |
| | 84.5 wt. %), Silica, | active | active | active |
| | Dimethicone | TiO₂) | TiO₂) | TiO₂) |
| B | Water | q.s. to 100 | q.s. to 100 | q.s. to 100 |
| | Sodium polyacrylate | 0.30 | 0.30 | 0.30 |
| | Glycerin | 2.00 | 2.00 | 2.00 |
| | Xanthan Gum | 0.20 | 0.20 | 0.20 |
| | Disodium EDTA | 0.20 | 0.20 | 0.20 |
| | Sun Protection Factor | 30.1 | 19.9 | 25.4 |
| | (SPF/calculated) | | | |
| | UVA1/UV ratio (calculated | 0.84 | 0.77 | 0.82 |
| | using the final extinction | | | |
| | spectrum available through | | | |
| | the export function of | | | |
| | the calculation) | | | |

The three compositions comparative formulations 1 and 2 and inventive formulation 3 were manufactured as follows:
Part A was heated to 80° C. while stirring without titanium dioxide. Once part A was homogenous, titanium dioxide was added, homogenized using an ultra turrax device until the titanium dioxide was well dispersed and part A was fully homogeneous.
Part B was heated to 80° C. while stirring and then part A was added to part B while stirring. The combined mixture was then homogenized using an ultra turrax device.

The formulation was cooled down to room temperature under stirring.

SPF and UVA1/UV ratio values for the formulations were calculated using in-silico determination methods using the BASF sunscreen simulator (https://sunscreensimulator.basf.com/). The BEMT-content drives the SPF value. The results show that inventive formulation 3 provides excellent sunscreen protection in the entire UV range. The inventive formulation also has a UV1/UV ratio of more than 0.7.

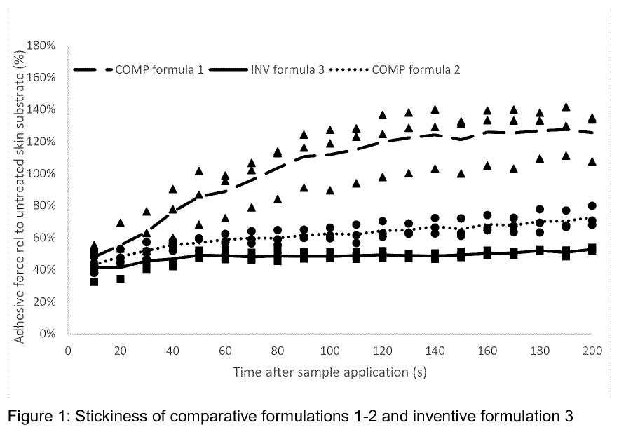

The three formulations were compared for their stickiness using the stickiness measurement described above. The results can be seen in Table 2 and FIG. 1. The absolute values for the three formulations are shown in Table 2. The normalized values against a control (untreated collagen membrane) are found in FIG. 1

TABLE 2
Stickiness of the three compositions in mN

| | Stickiness | Stickiness | Stickiness |
|---|---|---|---|
| | Comp. | Comp. | Inv. |
| | Form. 1 | Form. 2 | Form. 3 |
| 50 s after application | 59.8 mN | 29.3 mN | 28.9 mN |
| 100 s after application | 82.9 mN | 32.3 mN | 30.8 mN |
| 150 s after application | 94.7 mN | 34.8 mN | 32.9 mN |

Comparative formulation 1 which comprises BEMT and titanium dioxide as UV filters but is free of avobenzone exhibits the highest stickiness after 100 s. Therefore, comparative formulation 1 has a high stickiness that can result in an unpleasant feeling on the skin and adherence of particles like sand to the surface of the skin. Comparative formulation 2 which is free of BEMT has a lower stickiness after 100 s. This shows that BEMT is responsible for the stickiness of the formulation in comparative formulation 1. The combination of BEMT and avobenzone, however, reduces the stickiness of the composition even further than in both comparative formulations 1 and 2 (see FIG. 1). Inventive formulation 3 exhibits the lowest level of stickiness after 100 s after sample application when adjusting for the control (see FIG. 1) and similar levels of stickiness in absolute terms as comparative formulation 2. Therefore, the addition of avobenzone to a sunscreen or daily care composition comprising BEMT and titanium dioxide as UV filters leads to a surprising reduction in stickiness of the composition.

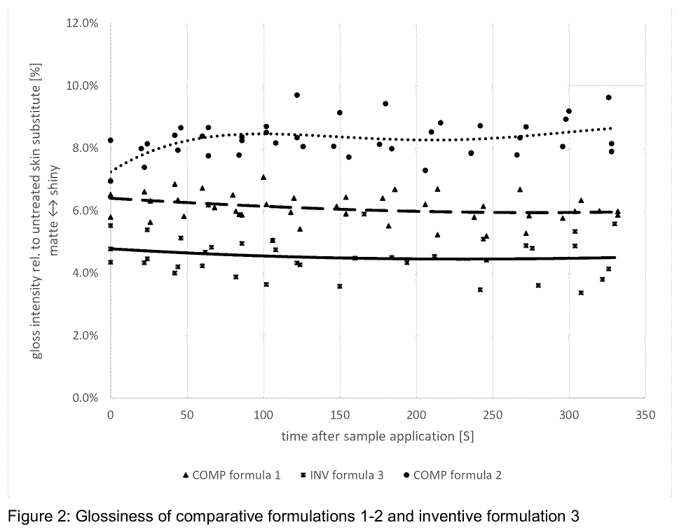

The glossiness of each of the three formulations was also tested. The results can be seen in FIG. 2. A lower glossiness of the sunscreen or daily care composition is generally preferred—it means that the sunscreen is optically neutral and is not shiny on the skin. The formulation comprising only avobenzone as organic UV filter showed an elevated glossiness level compared to the other two formulations. Interestingly, the inventive formulation comprising avobenzone and BEMT had a lower glossiness than both comparative formulations 1 and 2. Therefore, the inventive composition comprising a UV filter combination of BEMT, avobenzone and titanium dioxide exhibits a low stickiness as well as a low glossiness.

1. Sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone), and
(iii) titanium dioxide;
wherein the sunscreen or daily care composition is free of UV filters selected from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol] (bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (octinoxate), 2-ethylhexyl 2-cyano-3,3-diphenylprop-2-enoate (INCI octocrylene), and 2-hydroxy-4-methoxybenzophenone (oxybenzone).

2. Sunscreen or daily care composition according to claim 1, wherein the sunscreen or daily care composition is free of any further organic UV filter.

3. Sunscreen or daily care composition according to claim 1, wherein the sunscreen or daily care composition comprises phenylbenzimidazole sulfonic acid (Enzulizole).

4. Sunscreen or daily care composition according to claim 3, wherein the sunscreen or daily care composition is free of any further organic UV filter.

5. Sunscreen or daily care composition according to claim 1, wherein the sunscreen or daily care composition comprises zinc oxide.

6. Sunscreen or daily care composition according to claim 1, wherein the sunscreen or daily care composition is free of zinc oxide.

7. Sunscreen or daily care composition according to claim 1, wherein the titanium dioxide (iii) has a number-average elementary particle diameter of less than 1000 nm and/or is coated.

8. Sunscreen or daily care composition according to claim 1, wherein the weight ratio of (i) bis-ethylhexyloxyphenol methoxyphenyl triazine (bemotrizinol) to (ii) 1-(4-(1,1-dimethylethyl)phenyl)-3-(4-methoxyphenyl)propane-1,3-dione (avobenzone) is from 1:3 to 3:1.

9. Sunscreen or daily care composition according to claim 1, wherein the weight ratio of (i) bis-ethylhexyloxyphenol methoxyphenyl triazine (bemotrizinol) to (ii) 1-(4-(1,1-dimethylethyl)phenyl)-3-(4-methoxyphenyl)propane-1,3-dione (avobenzone) is 1:1.

10. Sunscreen or daily care composition according to claim 1, wherein the sunscreen or daily care composition comprises at least one compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, a fused ring cyanoacrylate derivative, polyester-25, butyloctyl salicylate and combinations thereof.

11. Sunscreen or daily care composition according to claim 1, wherein the sunscreen or daily care composition comprises
(i) bis-ethylhexyloxyphenol methoxyphenyl triazine (bemotrizinol) in an amount of 0.5 to 6.0 wt. %;
(ii) 1-(4-(1,1-dimethylethyl)phenyl)-3-(4-methoxyphenyl)propane-1,3-dione (avobenzone) in an amount of 1.0 to 5.0 wt. %;
(iii) titanium dioxide in an amount of 2.0 to 25.0 wt. %;
each based on the total weight of the sunscreen or daily care composition.

12. Sunscreen or daily care composition according to claim 1, wherein the sunscreen or daily care composition comprises
(i) bis-ethylhexyloxyphenol methoxyphenyl triazine (bemotrizinol) in an amount of 2.5 to 6.0 wt. %;
(ii) 1-(4-(1,1-dimethylethyl)phenyl)-3-(4-methoxyphenyl)propane-1,3-dione (avobenzone) in an amount of 2.5 to 5.0 wt. %;
(iii) titanium dioxide in an amount of 2.0 to 25.0 wt. %;
each based on the total weight of the sunscreen or daily care composition.

13. Sunscreen or daily care composition according to claim 1, wherein the sunscreen or daily care composition exhibits an adhesive force of less than 60 mN in an in-vitro adhesion test 100 s after application.

14. Sunscreen or daily care composition according to claim 1, wherein the sunscreen or daily care composition exhibits a reduced adhesiveness compared to a sunscreen or daily care composition comprising (i) bis-ethylhexyloxyphenol methoxyphenyl triazine (bemotrizinol) and (iii) titanium dioxide but being free of (ii) 1-(4-(1,1-dimethylethyl)phenyl)-3-(4-methoxyphenyl)propane-1,3-dione (avobenzone).

15. Sunscreen or daily care composition according to claim 1, wherein the sunscreen or daily care composition has a SPF of more than 15 or more than 20 or more than 25 and a UVA1/UV ratio of at least 0.7 and/or wherein the sunscreen or daily care composition has a photostability of more than 80%.

16. Sunscreen or daily care composition according to claim 1, wherein the sunscreen or daily care composition exhibits a reduced glossiness compared to a sunscreen or daily care composition comprising only one of (i) bis-ethylhexyloxyphenol methoxyphenyl triazine (bemotrizinol) and (ii) 1-(4-(1,1-dimethylethyl)phenyl)-3-(4-methoxyphenyl)propane-1,3-dione (avobenzone).

17. Method for reducing the adhesiveness of a sunscreen or daily care composition comprising
(i) bis-ethylhexyloxyphenol methoxyphenyl triazine (bemotrizinol) comprising the step of adding
(ii) 1-(4-(1,1-dimethylethyl)phenyl)-3-(4-methoxyphenyl)propane-1,3-dione (avobenzone) to the sunscreen or daily care composition.

18. Method for reducing the glossiness of a sunscreen or daily care composition comprising (ii) 1-(4-(1,1-dimethylethyl)phenyl)-3-(4-methoxyphenyl)propane-1,3-dione (avobenzone) comprising the step of adding (i) bis-ethylhexyloxyphenol methoxyphenyl triazine (bemotrizinol) to the sunscreen or daily care composition.

19. Sunscreen or daily care composition comprising a UV filter combination of (i) bis-ethylhexyloxyphenol methoxyphenyl triazine (bemotrizinol), (ii) 1-(4-(1,1-dimethylethyl)phenyl)-3-(4-methoxyphenyl)propane-1,3-dione (avobenzone) and (iii) titanium dioxide and being free of any further UV filter.
